(19) United States
(12) Patent Application Publication
(10) Pub. No.: US 2025/0295443 A1
(43) Pub. Date: Sep. 25, 2025

(54) SURGICAL INSTRUMENT WITH ADAPTABLE CLAMPING TIME

(71) Applicant: CILAG GMBH INTERNATIONAL, ZUG (CH)

(72) Inventors: ZHIJUN LIU, MASON, OH (US); PAUL MOUBARAK, WEST CHESTER, OH (US); DOUGLAS MARRIOTT, MAINEVILLE, OH (US); SHIV WADWANI, SAN JOSE, CA (US); MAXIMILIAN REESE, CINCINNATI, OH (US)

(21) Appl. No.: 18/609,075

(22) Filed: Mar. 19, 2024

(51) Int. Cl. A61B 18/08; A61B 17/00; A61B 17/072; A61B 90/00; G16H 40/63

(52) U.S. Cl. CPC A61B 18/085; G16H 40/63; A61B 2017/00398; A61B 2017/07257; A61B 2090/066

(57) ABSTRACT

Surgical instruments, robotic surgery systems, software for the same, and associated methods are disclosed in which instrument data is collected during a clamping time period in which tissue is clamped between opposing jaws. During the clamping time period, the instrument data includes a predictive portion in which the instrument data decays exponentially and therefore can be characterized by a mathematical feature such as a time constant, initial force or torque, elapsed decay time, etc. End time for the clamping time period, operational parameters for the instrument following the clamping time period, tissue characteristics, operational parameters for the instrument in successive clamping attempts, end effector articulation, and other instrument functions can be set/controlled based at least in part on the mathematical feature. In some examples, the instrument data includes a motor parameter which includes motor torque, clamping force, and/or motor speed.

### FIELD

This application relates generally to medical devices, and in particular to motor driven surgical staplers.

### BACKGROUND

Innovation in surgical stapling technology has evolved from manually operated handheld staplers, to handheld powered staplers, to robotic surgical staplers. Manual staplers clamp tissue, deliver staples, and drive a knife blade through mechanical force applied to lever(s) on a handle of the stapler. Powered staplers, including handheld and robotic surgical staplers, use an electrically powered motor to drive the knife blade and staples. Robotic surgical staplers, and some handheld powered staplers, also use an electrically powered motor to clamp tissue. Many surgical stapling challenges relate to tissue dynamics, tissue movement, and tissue variability. Different tissue types present unique tissue challenges. Achieving sufficient tissue compression facilitates proper staple delivery and cutting during a firing stroke. Various systems and methods for monitoring tissue compression for surgical stapling have been disclosed. For instance, U.S. Pat. No. 10,729,432; 11,013,528; 11,547,468; and 11,653,918 disclose example systems and methods for sensing tissue compression and controlling operation of the surgical stapler in response to tissue compression, each of which are incorporated by reference as if set forth in their entireties herein. In many present surgical stapler procedures, regardless of whether a mechanical or powered surgical stapler is used, protocol typically requires that the tissue is clamped for approximately 15 seconds to achieve sufficient tissue compression prior to a firing stroke.

Tissue compression is also a consideration in other surgical operations such as suturing, cauterization, and temporary clamping.

### SUMMARY

Examples disclosed herein generally describe powered surgical instruments and associated software for driving a motor in the powered surgical instrument to provide an adaptive clamping time period. Example surgical instruments are also described herein. As a motor closes jaws of the surgical instrument, instrument data such as clamping force and/or motor torque is tracked during a portion of a clamping time period in which the instrument data decays with time due to tissue relaxation. The decay is used to determine total clamping time needed to achieve sufficient tissue compression before initiating the next surgical operation. For instance, a robotic surgery system can include a stapler tool with motor-driven jaw clamping such that the force and/or torque decays as the tissue relaxes between the jaws. The software can cause the jaws to clamp tissue prior to a firing stroke for a time period that is determined based at least in part on the force and/or torque decay. The software can initiate a firing stroke of the stapler tool immediately after the clamping time period is elapsed. The software can further be configured to characterize the tissue based at least in part on the motor force and/or torque decays. The software may be applied to alternative surgical stapling tools such as handheld powered staplers. The software may be applied to alternative robotic surgical tools such as graspers, forceps, suture tools, and cauterization tools, and other such tools as understood by a person skilled in the pertinent art. In one example, a surgical instrument includes an end effector, a motor, and a motor control circuit. The end effector includes a pair of jaws. The motor assembly includes a motor mechanically coupled to the end effector. The motor assembly is configured to actuate the end effector to grasp and compress tissue between the pair of jaws. The motor control circuit is configured to electrically drive the motor during a clamping time period, monitor a motor parameter of the motor during the clamping time period, and determine an end time (t_end) of the clamping time period based at least in part on the motor parameter.

In another example, a surgical instrument includes an end effector, a motor assembly, and a motor control circuit. The end effector includes a pair of jaws. The motor assembly includes a motor mechanically coupled to the end effector. The motor assembly is configured to actuate the end effector to grasp and compress tissue between the pair of jaws. The motor control circuit is configured to electrically drive the motor during a plurality of short duration clamping time periods at a plurality of strain rates, monitor a motor parameter of the motor during the plurality of clamping time periods, and determine a tissue parameter based at least in part on the motor parameter during the plurality of clamping time periods.

### BRIEF DESCRIPTION OF THE DRAWINGS

While the specification concludes with claims, which particularly point out and distinctly claim the subject matter described herein, it is believed the subject matter will be better understood from the following description of certain examples taken in conjunction with the accompanying drawings, in which like reference numerals identify the same elements. The figures depict one or more implementations of the inventive devices, by way of example only, not by way of limitation.

FIG. 1 is a perspective view of an exemplary surgical stapler tool of a robotic surgery system.

FIG. 2 is a perspective view of an exemplary handheld powered surgical stapler.

FIG. 3 is an illustration of an exemplary end effector of an exemplary powered surgical stapler prior to compression of tissue between jaws.

FIGS. 4A and 4B are illustrations of the exemplary end effector at an early stage of compression of tissue between the jaws.

FIGS. 5A and 5B are illustrations of the exemplary end effector at the end of the clamping time period.

FIG. 6 is a block diagram of an exemplary end effector drive system for a powered surgical instrument.

FIG. 7 is a chart illustrating decay of force/torque during a portion of a clamping time period in which tissue is relaxing.

FIG. 8 is a flow diagram of a method for a stapling procedure that includes clamping tissue for a clamping time period and deploying staples during a firing stroke following the clamping time period.

FIG. 9 is a flow diagram of another method for controlling clamping time during a clamping time period.

FIG. 10 is a chart illustrating predicted force correlated with measured force using a learned/curve fitted exponential decay model.

FIG. 11 is a chart illustrating transformed peak firing force as a function of a transformed input feature using a learned/curve fitted exponential decay model.

FIG. 12 illustrates a correlation matrix and raw data plotting of key features extracted from decay of force/torque during a predictive portion of the clamping time period.

FIG. 13 is an illustration of an example end effector in an open position.

FIGS. 14A, 14B, 14C, and 14D are a sequence of illustrations of a clamping mechanism of an end effector during a clamping time period.

FIG. 15 is an illustration of the example end effector of FIG. 13 in a closed position.

FIG. 16 is a flow diagram of another method for controlling clamping time during a clamping time period.

### DETAILED DESCRIPTION

The following detailed description should be read with reference to the drawings, in which like elements in different drawings are identically numbered. The drawings, which are not necessarily to scale, depict selected embodiments and are not intended to limit the scope of the invention. The detailed description illustrates by way of example, not by way of limitation, the principles of the invention. This description will clearly enable one skilled in the art to make and use the invention, and describes several embodiments, adaptations, variations, alternatives, and uses of the invention, including what is presently believed to be the best mode of carrying out the invention.

As used herein, the terms “about” or “approximately” for any numerical values or ranges indicate a suitable dimensional tolerance that allows the part or collection of components to function for its intended purpose as described herein. More specifically, “about” or “approximately” may refer to the range of values +10% of the recited value, e.g., “about 90%” may refer to the range of values from 81% to 99%.

As used herein, the terms “patient,” “host,” “user,” and “subject” refer to any human or animal subject and are not intended to limit the systems or methods to human use, although use of the subject invention in a human patient represents a preferred embodiment. As well, the term “proximal” indicates a location closer to the operator whereas “distal” indicates a location further away to the operator or physician.

As used herein, the term “memory” and “non-transitory computer-readable media” are used interchangeable and are understood to include, but are not limited to, random access memory (RAM), read-only memory (ROM), electronically erasable programmable ROM (EEPROM), flash memory or other memory technology, compact disc ROM (CD-ROM), digital versatile disks (DVD) or other optical storage, magnetic cassettes, magnetic tape, magnetic disk storage or other magnetic storage devices, or any other tangible, physical medium which can be used to store computer readable information.

Alternative apparatus and system features and alternative method steps are presented in example embodiments herein. Each given example embodiment presented herein can be modified to include a feature and/or method step presented with a different example embodiment herein where such feature and/or step is compatible with the given example as understood by a person skilled in the pertinent art as well as where explicitly stated herein. Such modifications and variations are intended to be included within the scope of the claims.

Examples presented herein are generally directed to surgical instruments, robotic surgery systems, software for the same, and associated methods in which instrument data is collected during a clamping time period in which tissue is clamped between opposing jaws. The instrument data is utilized for various instrument functions such as adaptively determining an end time for the clamping time period, determining operational parameters for the instrument following the clamping time period, determining tissue characteristics, determining operational parameters for the instrument in successive clamping attempts, improving end effector articulation, etc. During the clamping time period, the instrument data includes a predictive portion in which the instrument data decays exponentially and therefore can be characterized by a time constant, initial torque or force, elapsed decay time, and/or other mathematically-derived features as understood by a person skilled in the art. The end time for the clamping time period, operational parameters for the instrument following the clamping time period (e.g. aspects of the firing control sequence), tissue characteristics, operational parameters for the instrument in successive clamping attempts, end effector articulation, and other instrument functions can be set/controlled based at least in part on the mathematically derived feature(s) (e.g., time constant) of the instrument data during the clamping time period.

The disclosure focuses on surgical staplers with motor-driven tissue clamping such that motor torque decays exponentially during the predictive portion of the clamping time period. A mathematical feature of the motor torque (e.g., exponential decay time constant, initial torque or force, elapsed decay time, etc.) is utilized to for various instrument functions, including those listed in the preceding paragraph. As discussed herein, and as understood to a person skilled in the pertinent art, the principles of utilizing a mathematical feature (e.g., exponential decay time constant, initial torque or force, elapsed decay time, etc.) of instrument data obtained during a clamping time period of a surgical instrument to affect various instrument functions can be broadly applied to a variety of surgical instruments.

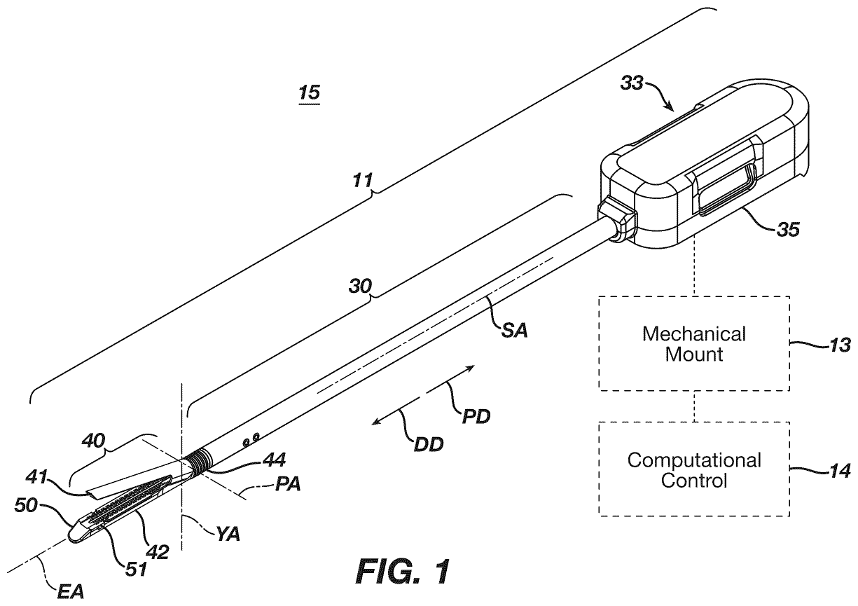

FIG. 1 is a perspective view of an exemplary surgical stapler tool 11 of a robotic surgery system 15. The surgical stapler tool 11 can be mounted to a mechanical mount 13, such as robotic arm cart, that can be controlled by a computational control unit 14, such as a controller station, of a robotic surgical system 15. An example robotic surgical system is disclosed in U.S. Pat. No. 7,524,320, incorporated herein by reference. The surgical stapler tool 11 can be used with various alternative robotic surgical systems as understood by a person skilled in the pertinent art. The surgical stapler tool 11 can be modified for use with such robotic surgical systems as understood by a person skilled in the pertinent art.

The surgical stapler tool 11 includes a stapler portion 12 and a mounting portion 33. The stapler portion 12 extends in a distal direction DD from the mounting portion 33 and is configured to perform surgical operations on a patient. The mounting portion 33 is configured to mount to the mechanical mount 13 of the surgical system. As illustrated, the mounting portion 33 includes a release mechanism 34 that can be operated by hand to detach the mounting portion 33 from the mechanical mount 13. The mounting portion 33 includes a housing 35 covering mechanical mechanisms of the mounting portion 33 and configured to mate the mechanical mechanisms of the mounting portion 33 to the mechanical mount 13 of the surgical system.

The stapler portion 12 includes a shaft 30 that is sized, shaped, and otherwise configured to extend through a body opening of the patient. The end effector 40 is configured deliver staples 51. The end effector 40 may also be configured to cut tissue within the body of the patient. The end effector 40 includes an anvil 41 and a staple jaw 42 opposite the anvil 41. The anvil 41 and staple jaw 42 are collectively referred to herein as “jaws.” The staple jaw 42 can include a staple cartridge 50 containing the staples 51. The staple cartridge 50 can be replaceable. Alternatively, the end effector 40 may be replaceable. The anvil 41 and staple jaw 42 are illustrated in an open position. The anvil 41 and staple jaw 42 can be moved toward each other to move the end effector 40 to a clamped configuration. The end effector 40 can be actuated to deploy staples 51 into tissue during a firing stroke. Rotation of the anvil 41 to clamp tissue and deployment of staples 51 during a firing stroke are respectively motor driven by one or more motors.

The computational control 14 is configured to actuate mechanisms of the mechanical mount 13, which in turn, position the surgical stapler tool 11 and interface with mechanical controls of the mounting portion 33 of the surgical stapler 11 to operate the stapler portion 12. The stapler portion 12 may be driven by one or more motors which may be located in the housing 35, the mechanical mount 13, or elsewhere in the robotic surgical system 15. Torque of motor(s) located outside of the mounting portion 33 are transmitted via the mechanical mount 13 to the mounting portion 33 of the surgical stapler tool 11 by mechanical interconnect(s) between the mechanical mount 13 and mounting portion 33. The torque of motor(s) within the mounting portion 33 and/or transmitted to the mounting portion 33 via the mechanical mount 13 are transmitted by elongated mechanical structures through the shaft 30 to the end effector 40.

The surgical stapler tool 11 may be purely mechanical or may include electronic components such as motors, processors, memory, etc.

Software to control tissue clamping by the end effector 40 can be stored in memory of the computational control 14 and/or in memory of the surgical stapler tool 11. The software can be configured to monitor an instrument parameter during a clamping time period and utilize the instrument parameter data for various instrument functions. For instance, the software can determine clamping time based on clamping force and/or torque of motor(s) driving the end effector 40 during clamping. The software can otherwise include instructions for operating the end effector 40 as described in greater detail elsewhere herein.

The surgical stapler tool 11 further includes an articulation joint 44 between the shaft 30 and the end effector 40. The articulation joint 44 is configured to permit the end effector 40 to be angled in relation to a longitudinal axis S-A of the shaft 30. As illustrated, the end effector 40 has a longitudinal axis EA that is aligned with the shaft axis S-A. The articulation joint can be bent so that the end effector axis EA is angled toward a pitch axis PA, yaw axis YA, or some combination thereof. The articulation joint 44 can be bent manually by pressing the end effector 40 against tissue or other object or powered via one or more motor(s) of the robotic surgical system 15. Additionally, or alternatively, the articulation joint 44 can be powered by the same or different motor configured to actuate clamping and/or firing of the end effector 40. The robotic surgical system 15 can include an articulation control circuit configured to modify the angle of the end effector 40 based at least in part on a motor parameter of the motor driving the end effector 40 during the clamping time period. This can be advantageous in systems in which the end effector 40 is articulated by an articulation load. The articulation cable load can be improved during a firing stroke by either tightening or relaxing the cable through the articulation joint 44 depending on various conditions such as articulation joint stability (e.g., risk of de-articulation) and/or high cable load propagation. The articulation control circuit can be located in the computational control 14, the mounting portion 35, or elsewhere in the robotic surgical system 15.

The surgical stapler tool 11 can be modified to provide additional or alternative therapeutic treatments involving clamping tissue by the end effector 40. For instance, the end effector 40 can be modified to include electrodes configured to delivery thermal treatment to tissue in addition to, or in lieu of staples 51. The modified tool 11 can be driven by software including methods for monitoring force/torque response characteristics during jaw clamping, assuming the forces/torques experienced during jaw closure can be measured/monitored in real-time.

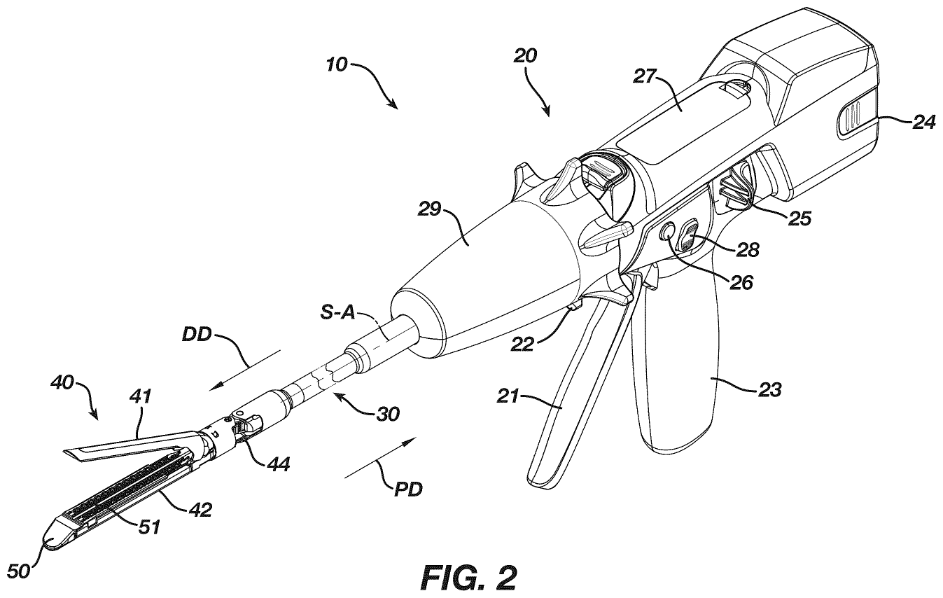

FIG. 2 is a perspective view of an exemplary handheld powered surgical stapler 10 including a handle 20, a shaft 30, and an end effector 40. The handle 20 is configured to be grasped, manipulated, and actuated by a clinician. The shaft 30 is sized, shaped, and otherwise configured to extend through a body opening of the patient. The end effector 40 is configured deliver staples 51. The end effector 40 may also be configured to cut tissue within the body of the patient. The end effector 40 and shaft 30 of the surgical stapler 10 illustrated in FIG. 2 can be configured similar to the end effector 40 and shaft of the surgical stapling tool 11 illustrated in FIG. 1.

The handle 20 can include a closure trigger 21, a firing trigger 22, and a grip 23 sized such that a clinician can single-handedly hold the surgical stapler 10 by the grip 23 while manipulating the closure trigger 21 or the firing trigger 22. The closure trigger 21 is operably connected to a motor disposed within the handle 20 such that when the closure trigger 21 is pulled, the motor is driven to cause the end effector 40 to clamp tissue. The firing trigger 22 is operably connected to a motor disposed within the handle 20 such that when the firing trigger 22 is pulled, the motor is driven to cause the end effector 40 to deploy staples 51 into the clamped tissue and may also cut the clamped tissue. The closure trigger 21 and the firing trigger 22 can be coupled to separate respective motors, or the same motor.

The handle 20 can further include additional features such as a firing trigger lock mechanism (not illustrated) which can be manipulated to prevent actuation of the firing trigger 22, a power pack 24 configured to provide electrical power to the motor and other electrical components of the powered surgical stapler 10, a closure release button 25 which can be manipulated to release the end effector 40 and the closure trigger 21 from the clamped position, a home button 26 that can be pressed to cause the motor to move a firing assembly in the proximal direction PD to a home position, a manual override 27 including a mechanical actuator which can be manipulated to mechanically move the firing assembly proximally to the home position, articulation buttons 28 that can be pressed to cause a motor to articulate the end effector 40 at an articulation joint 44 so that the end effector 40 is at an angle with a longitudinal axis S-A of the shaft 30, a rotatable nozzle 29 configured to be rotated so that the shaft 30 and end effector rotate about the shaft axis S-A, a display (not illustrated) configured to display information related to the surgical stapler, variations thereof, other compatible features of a powered surgical stapler handle, and combinations thereof.

The end effector 40 includes an anvil 41 and a staple jaw 42 opposite the anvil 41. The anvil 41 and staple jaw 42 are illustrated in an open position. The anvil 41 and staple jaw 42 can be moved toward each other to move the end effector 40 to a clamped configuration. For instance, tissue (not illustrated) can be positioned between the anvil 41 and staple jaw 42 in the open position, and the anvil 41 can rotate toward the staple jaw 42 to clamp the tissue.

When the end effector 40 is in the clamped configuration, the firing trigger 22 can be pulled to cause deployment of staples 51 from the cartridge 50 and may also cause cutting of tissue.

Software to control tissue clamping by the end effector 40 can be stored in memory of the handheld powered surgical stapler 10. The software can be configured to determine clamping time based on force and/or torque of motor(s) driving the end effector 40 during clamping. The software can be configured to provide user feedback and/or control operation of the surgical stapler based on the clamping time. For instance, the software can be configured to provide a visual display of clamping time (e.g. a count-down to the end of the clamping time period) so that the clinician is alerted when the tissue is sufficiently compressed to engage a firing stroke. Additionally, or alternatively, the software may lock-out the firing trigger 22 so that the clinician is unable to initiate the firing stroke until after the end of the clamping time period. Additionally, or alternatively, the software can be configured to automatically initiate a firing stroke at the end of the clamping time period. The surgical stapler 10 can include an articulation control circuit configured to modify the angle of the end effector 40 at the articulation joint 44 based at least in part on a motor parameter of the motor driving the end effector 40 during the clamping time period. The software can otherwise include instructions for operating the end effector 40 as described in greater detail elsewhere herein.

Portions of the surgical stapler 10 may be detachable and interchangeable. Staples 51 may be housed in a staple cartridge 50 that is detachable from the end effector 40. The end effector 40 may be detachable from the shaft 30, and the shaft 30-handle 20 combination may be configured for use in connection with interchangeable end effectors. At least a portion of the shaft 30 including the end effector 40 may be detachable from the handle 20, and the handle 20 may be configured for use in connection with interchangeable shaft assemblies having different shaft lengths and/or different end effectors attached thereto.

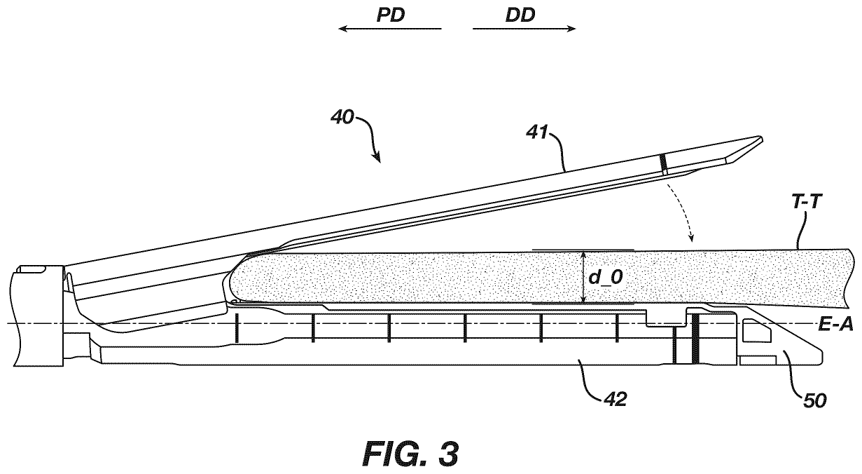

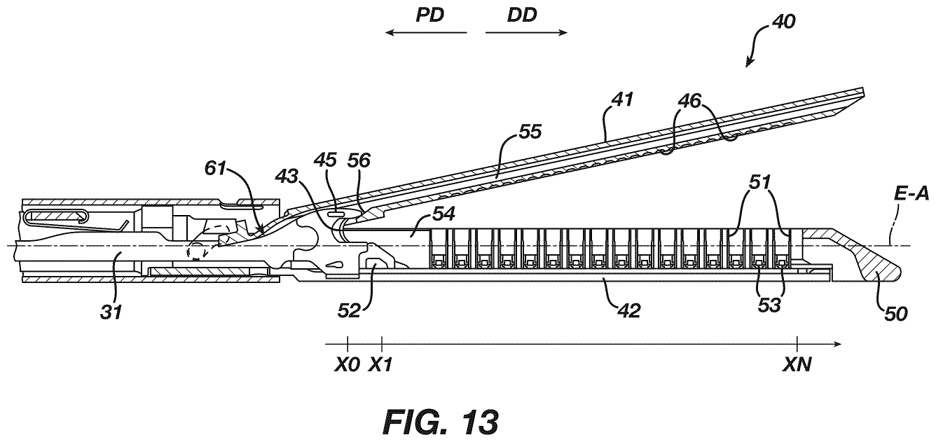

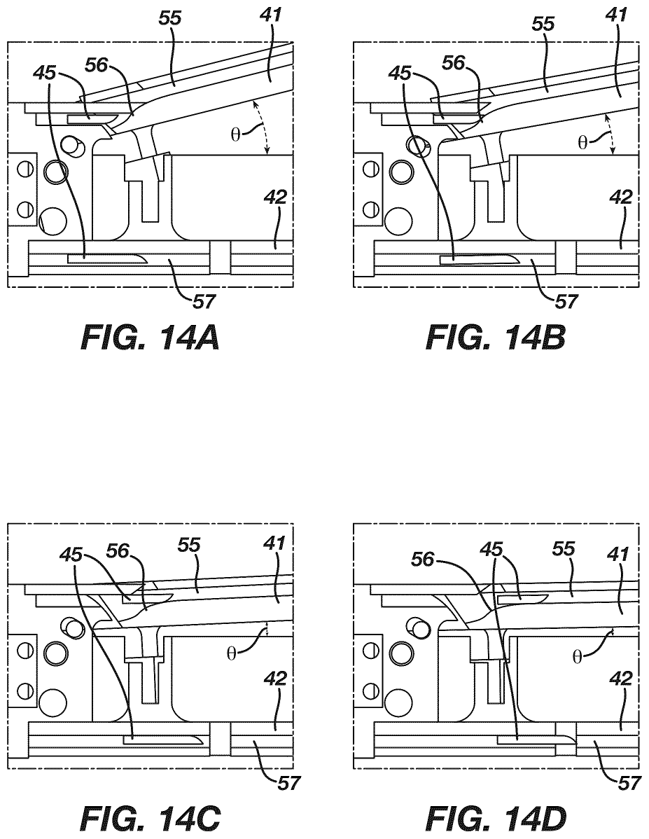

FIG. 3 is an illustration of an exemplary end effector 40 of an exemplary powered surgical stapler prior to compression of tissue TT between jaws. The end effector 40 can be configured for a robotic surgical system such as illustrated in FIG. 1, or a handheld surgical stapler 10 such as illustrated in FIG. 2. The end effector 40 generally presents an example end effector of a surgical instrument configured for compression of tissue. The end effector 40 need not deploy staples and may be adapted for transection, suturing, cauterization, temporary tissue clamping (e.g. graspers, bipolar, etc.), or other operations as understood by a person skilled in the pertinent art. The end effector 40, modified for alternative applications, may be configured for use with a robotic surgical system and/or handheld surgical device as understood by a person skilled in the pertinent art. For instance, the end effector 40 can be modified to include electrodes configured to delivery thermal treatment to tissue in addition to, or in lieu of staples 51. The end effector 40 can be driven by software including methods for monitoring force/torque response characteristics during jaw clamping, assuming the forces/torques experienced during jaw closure can be measured/monitored in real-time. FIGS. 13 and 14A through 14D illustrate some features of the example end effector 40 in greater detail as non-limiting examples.

The staple jaw 42 of the end effector 40 is aligned along a longitudinal axis E-A of the end effector 40. The tissue has an initial thickness d_0 prior to being clamped. The rotation of the anvil 41 toward the staple jaw 42 is motor driven.

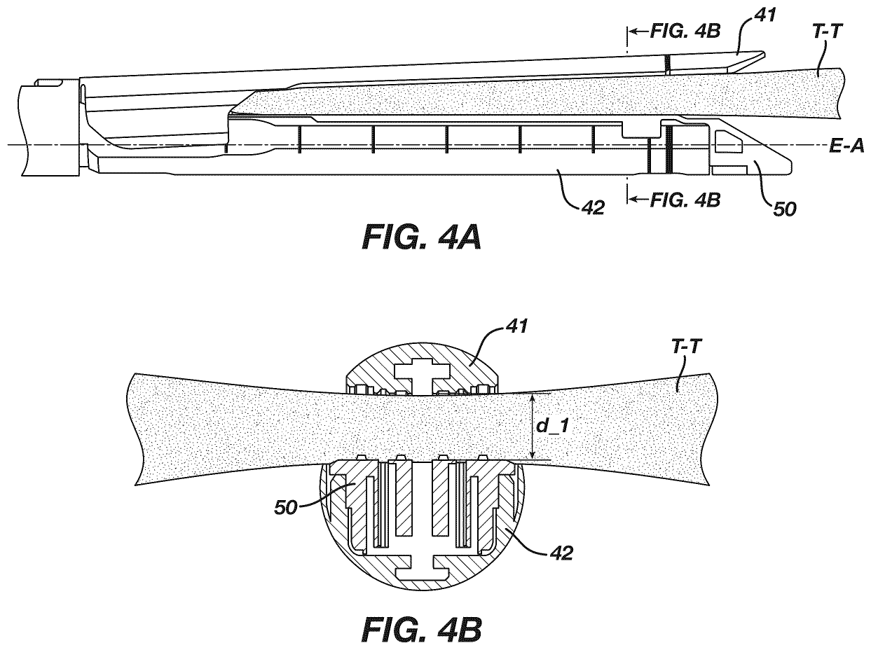

FIGS. 4A and 4B are illustrations of the exemplary end effector 40 at an early stage of compression of tissue TT between the anvil 41 and staple jaw 42. FIG. 4B is a cross-sectional view through the anvil 41, staple jaw 42, and tissue TT as indicated in FIG. 4A. As the anvil 41 is rotated by motor torque/force toward the staple jaw 42 during the clamping time, the tissue TT is compressed. The tissue thickness d_1 at an early stage of the clamping time period is less than the initial thickness d_0.

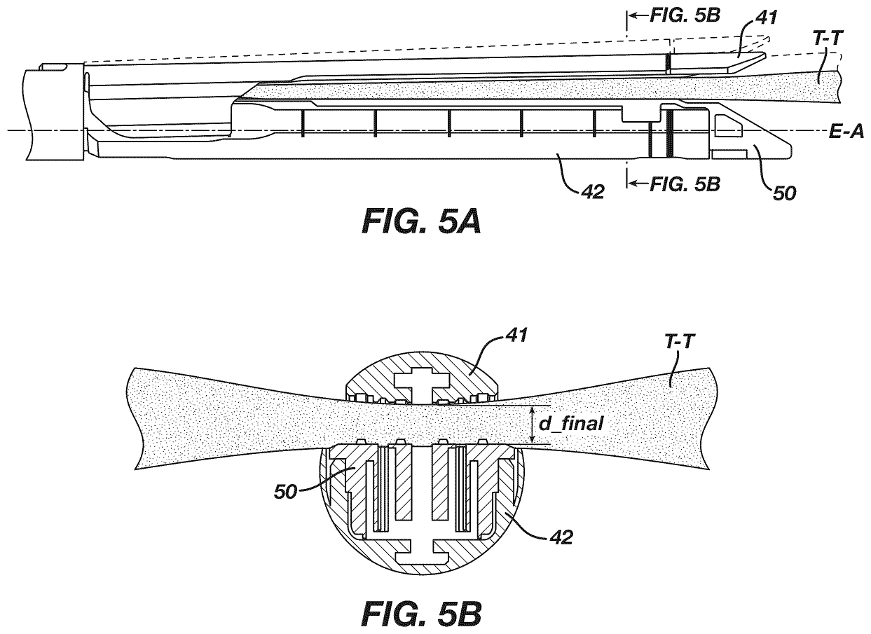

FIGS. 5A and 5B are illustrations of the exemplary end effector 40 at the end of the clamping time period. The tissue thickness d_final at the end of the clamping time period is reduced so that subsequent surgical operations can be performed, such as initiating a firing stroke of a surgical stapler. In the illustrated example, the final tissue thickness d_final is approximately 0.5 mm less than the initial tissue thickness d_0 prior to the clamping time period as measured approximate a distal end of the treated tissue in the end effector 40.

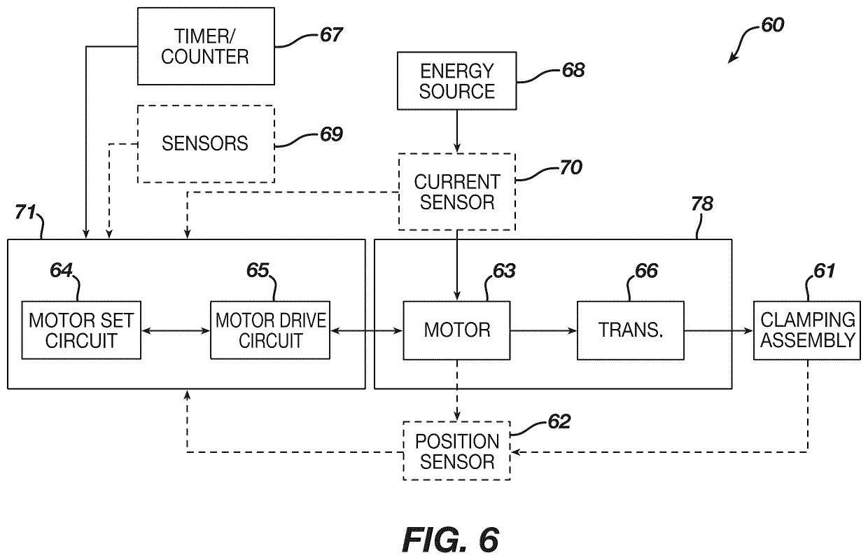

FIG. 6 is a block diagram of an exemplary end effector drive system 60 for a powered surgical instrument. The end effector drive system 60 is configured to performed powered actuation of the end effector 40, including clamping of tissue. The end effector drive system 60 is configured to actuate the clamping assembly 61 to close the jaws 41, 42 of the end effector 40 illustrated in FIG. 3 and variations thereof as described herein and otherwise understood by a person skilled in the pertinent art. The end effector drive system 60 may further be configured to perform powered actuation of additional surgical operations of the end effector 40 such as driving a firing assembly to deploy staples 51.

The end effector drive system 60 includes a motor control circuit 71 configured to drive a motor 63. The end effector drive system 60 includes a transmission 66 configured to convert the rotational movement of a rotor of the motor 63 into longitudinal movement of a clamping assembly 61. The motor 63 and transmission 66 are collectively referred to herein as a motor assembly 78. Examples of clamping assemblies 61 are illustrated in FIGS. 13 and 14A through 14D.

In some examples, clamping and firing of the end effector 40 are both actuated by the same motor 63 and common mechanical features such as an I-beam 45 (FIGS. 13 and 14A through 14D) coupled to an elongated firing bar 31 (FIG. 13). In such examples, the clamping assembly 61 is also referred to as a firing assembly or a clamping/firing assembly. Alternatively, clamping and firing are actuated by separate motors and have distinct mechanical features. In some examples, the end effector drive system 60 is not configured for firing. Note that the motor 63 as illustrated, may represent more than one motor. The position, movement, displacement, and/or translation of one or more components of the clamping assembly 61, can be measured by one or more position sensors 62. The position sensor(s) 62 may be configured to detect movement of the clamping assembly 61 and/or rotation of the rotor of the motor 63. The position sensor(s) 62 can additionally or alternatively be configured to sense displacement of a clamping/firing assembly during a firing stroke.

The motor control circuit 71 is illustrated as including a motor set circuit 64 and motor drive circuit 65, which are illustrated as two separate blocks. The motor set circuit 64 and motor drive circuit 65 may be separate circuits or may be integrated as a single circuit. The motor set circuit 64 is configured to provide a motor setpoint signal output to the motor drive circuit 65. The motor setpoint signal is indicative of a target parameter, such as a target speed of the clamping assembly 61. The motor controller 65 is configured to provide a motor drive signal to the motor 63 such that the motor drive signal is based on the motor setpoint signal and intended to drive the motor 63 so that the clamping assembly 61 is driven to the target parameter.

The motor set circuit 64 and the motor drive circuit 65 may include one or more processors and memory (i.e., one or more non-transitory computer-readable medium) with instructions that can be executed by the one or more processors to cause the motor set circuit 64 and the motor drive circuit 65 to drive the motor 63. The motor set circuit 64 and/or motor drive circuit 65 can include a feedback controller, which can be one of any feedback controllers, including, but not limited to a PID, a State Feedback, LQR, and/or an Adaptive controller, for example. The motor set circuit 64 and/or motor drive circuit 65 can include a power source to convert the signal from the feedback controller into a physical input such as a constant voltage, pulse width modulated (PWM) voltage, frequency modulated voltage, current, torque, and/or force, for example.

The motor drive circuit 65 is configured to electrically drive the motor 63 during at least a portion of the clamping time period. The motor set circuit is configured to monitor a motor parameter of the motor during the clamping time period. The motor set circuit 64 is configured to determine an end time of a clamping time based at least in part on the motor parameter. Additionally, or alternatively, the motor set circuit 64 is configured to determine the end time of the clamping time period based on clamping force on tissue compressed by the end effector. The end effector drive system 60 can optionally include one or more sensor(s) 69 that can be configured to measure clamping force directly. For instance, sensor(s) 69 may be positioned on the end effector 40 to measure applied force and/or other tissue properties in a region of clamped tissue.

Configured as such, the motor control circuit 71 is configured to clamp tissue to achieve sufficient tissue compression so that a subsequent surgical operation can be performed such as deployment of staples from an end effector of a surgical stapler. The motor drive circuit 65 is configured to power the motor 63 to facilitate tissue compression during the clamping time period. The motor set circuit 64 is configured to determine the end time of the clamping time period.

In some examples, the motor parameter is correlated to clamping force, and the motor set circuit 64 is configured to estimate clamping force based at least in part on the motor parameter. For instance, the motor parameter can include motor torque, clamping force, and/or motor speed. Motor torque can be determined based at least in part on motor current from an energy source 68 sensed by a current sensor 70 as understood by a person skilled in the pertinent art. The motor assembly 78 can include an encoder or other suitable device to measure motor speed. Motor torque can be determined based at least in part on motor speed as understood by a person skilled in the pertinent art. Motor torque can be determined based at least in part on additional parameters, such as motor voltage, as understood by a person skilled in the pertinent art.

In some examples, the motor parameter is configured to exponentially decay through a predictive portion of the clamping time period. The firing driver 60 includes a timer/counter circuit 67 configured to provide an output signal, such as elapsed time or a digital count, to the motor set circuit 64. A time constant of the exponential decay of the motor parameter can be calculated based at least in part on information provided by the timer/counter circuit 67. The motor set circuit 64 can be configured to determine the end time of the clamping time period based at least in part on the time constant of the exponential delay of the motor parameter. In some examples, the motor set circuit 64 is configured to determine the end time of the clamping time period based at least in part on an exponential decay of motor torque. Additionally, or alternatively, the timer/counter circuit 67 may be utilized to determine timing of other mathematical features of the motor parameter such as an initial torque or force or elapsed decay time.

The end effector drive system 60 can be adapted for a robotic surgical system 15 or a handheld surgical device 10 such as illustrated in FIGS. 1 and 2. In one example robotic surgical system 15, portions or all of the timer/counter 67, portions or all of the energy source 68, portions or all of the current sensor 70, and portions or all of the motor control circuit 71 may be physically located in the computational control 14; portions or all of the motor assembly 78 may be physically located in the mechanical mount 13; and portions or all of the sensors 69, portions or all of the position sensor 62, and portions or all of the clamping assembly 61 may be physically located in the surgical stapler tool 11. In one example handheld powered surgical stapler 10, portions or all of the energy source 68, portions or all of the current sensor 70, portions or all of the motor control circuit 71, portions or all of the timer/counter 67, and portions or all of the motor assembly 78 are disposed in the handle of the handle 20; portions or all of the clamping assembly 61 extend through the shaft 30 to the end effector 40; and portions or all of the sensors 69 and position sensors 62 are disposed in the end effector 40. As understood by a person skilled in the pertinent art, the physical location and physical structure of each of the components of the end effector drive system 60 have several suitable possibilities not listed herein for the sake of brevity.

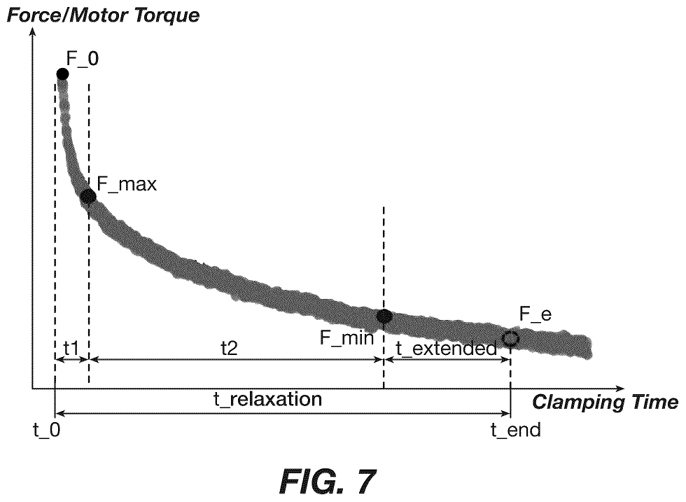

FIG. 7 is a chart illustrating decay of clamping force and/or motor torque during tissue relaxation time period t_relaxation of a clamping time period. The tissue relaxation time period may be preceded by a portion of the clamping time period in which the end effector is closing on tissue. Typically, closure of the end effector is about a second or less, while the tissue relaxation time period t_relaxation may be about 15 seconds. An object of the present invention is to vary the tissue relaxation time period t_relaxation, and therefore clamping time period, based on measuring electrical signals indicative of tissue properties during the tissue relaxation time period t_relaxation.

The beginning of the tissue relaxation time period may t_0 may occur when the jaws of the end effector have reached a predetermined angle and/or the motor driving end effector closure has made a predetermined rotation. The chart assumes that clamping force and motor torque are related and each respectively experience exponential decay. The chart presents an embodiment in which the motor parameter used to determine the clamping time period is motor torque. Motor torque may be measured directly (e.g., by torque sensors) or indirectly (e.g., calculated based on motor speed and power) as understood by a person skilled in the pertinent art. Alternatively, clamping force may be measured directly or determined by some other motor parameter which correlates to clamping force. Regardless, the clamping assembly 61 can be coupled to the motor assembly 78 (FIG. 6) such that the clamping force and/or the motor parameter decays exponentially during at least a portion of the clamping time period. In an alternative embodiment, a similar analysis may be performed based on plot of motor speed as a function of time.

The relaxation time period t_relaxation of the clamping time period is divided into three portions: an initial portion t1, a predictive portion t2, and a final portion t_extend. Prior to the relaxation time period t_relaxation, the motor control circuit 71 drives the motor 63 to engage the clamping assembly 61 and begin closure of the jaws 41, 42 of the end effector 40. The initial portion t1 starts at a start time t_0 of the relaxation time period t_relaxation. During the initial portion t1, the clamping force and/or motor torque is likely to experience transients due to engagement of the motor 63, tissue TT, and components of the clamping assembly 61. Instrument data during the initial portion t1 can be ignored. The initial portion t1 can be set to have a predetermined duration. Alternatively, the end of the initial portion t1 can triggered based on an instrument parameter such as reaching a predetermine threshold value (e.g. torque/force value F1_max illustrated in FIG. 7).

Referring collectively to FIGS. 6 and 7, the motor control circuit 71 continues to drive the motor 63 through the predictive portion t2 of the relaxation time period t_relaxation of the clamping time period. The motor control circuit 71 can be configured to calculate a time constant (or alternative mathematical feature) of the motor torque, clamping force (or alternative motor parameter) during the predictive portion t2. The predictive portion t2 begins after the initial transients in motor torque and clamping force have passed. The predictive portion t2 can be characterized as a portion of the clamping time period that fits well to a curve with an exponential decay. During the predictive portion t2 of the clamping time period, the motor torque (and/or clamping force and/or alternative motor parameter) starts at an initial maximum value F1_max and falls to a lower value F_min. Mathematical processes, such as curve fitting, can be utilized to determine the time constant (or initial torque, elapsed decay time, and/or other mathematical feature) of the motor torque, clamping force, and/or alternative motor parameter during the predictive portion t2.

The motor control circuit 71 is configured to determine the duration of the final portion t_extend based at least in part on the mathematical feature (e.g., time constant). For instance, the motor control circuit 71 may be configured to estimate an elapsed time required so that the motor torque (and/or clamping force and/or alternative motor parameter) falls from the lower value F_min to a predetermined threshold value F_e. This estimated elapsed time can be based on curve fitting or other mathematical process as understood by a person skilled in the pertinent art. Additionally, or alternatively, the motor control circuit 71 can be configured to compare measured motor torque during the final portion t_extend to a motor torque threshold F_e and determine the end time t_end of the clamping time period based at least in part on the comparison. Compared to prior operational procedures which rely on a fixed clamping time period (e.g. 15 seconds), the adaptive clamping time period may provide for faster procedures by eliminating unnecessary wait time for compliant tissue. The adaptive clamping time period may also result in more consistent tissue compression, which in turn can provide better outcomes for operations following the clamping time period (e.g., consisting firing strokes).

Continuing to refer collectively to FIGS. 6 and 7, in some examples, the clamping assembly 61 is configured as a clamping/firing assembly. In such examples, the motor assembly 78 is configured to drive the clamping/firing assembly 61 to clamp tissue during the clamping time period and also drive the clamping/firing assembly 61 during a firing stroke. In such examples, the motor control circuit 71 may be configured to estimate a peak firing force expected to be experienced during the firing stroke based at least in part on the motor torque (and/or clamping force and/or alternative motor parameter) monitored during the clamping time period. The motor control circuit 71 may be configured to compare the estimated peak firing force to a firing force threshold and determine the end time t_end of the clamping time period based at least in part on the comparison.

In some examples, the motor control circuit 71 is configured to initiate a firing stroke in response to the clamping time period reaching the end time t_end. This can be facilitated in examples in which the clamping assembly 61 is configured as a clamping/firing assembly and also in which the clamping assembly 61 is distinct from the firing assembly. The motor control circuit 71 can be configured to set a parameter of the firing stroke based at least in part on motor torque (and/or clamping force and/or alternative motor parameter) measured during the clamping time period. The parameter of the firing stroke set by the motor control circuit 71 can include a target acceleration and/or a target velocity of the clamping/firing assembly 61 (or distinct firing assembly) during the firing stroke. The firing stroke can include multiple segments over a firing stroke length such that the segments can have differing parameters. The motor control circuit 71 can be configured to set respective differing parameters for respective segments of the firing stroke. Trajectory of the clamping/firing assembly 61 (or distinct firing assembly) during a firing stroke (e.g., acceleration, velocity, multi-segment planning, compliance) can be modified, by the motor control circuit 71, as a function of the exponential fit parameters and/or sensed clamp force during the clamping time period. For instance, acceleration of the clamping/firing assembly 61 (or distinct firing assembly) during a firing stroke and initial target velocity of the clamping/firing assembly 61 (or distinct firing assembly) during the firing stroke can be informed by the decayed motor torque (and/or clamping force and/or alternative motor parameter) during the clamping time period prior to the firing stroke. The motor control circuit 71 can be configured to set additional and/or alternative firing stroke parameters as understood by a person skilled in the pertinent art. For instance, U.S. Pat. 10,307,717, incorporated by reference herein discloses a method for control of motor velocity of a surgical stapling and cutting instrument. Firing stroke parameters disclosed in U.S. Pat. No. 10,307,717, and other suitable firing stroke parameters as understood by a person skilled in the pertinent art may be set by the motor control circuit 71 based at least in part on motor torque (and/or clamping force and/or alternative motor parameter) measured during the clamping time period.

In some embodiments, the motor control circuit 71 is configured to estimate a tissue property based at least in part on motor torque (and/or clamping force and/or alternative motor parameter) during the clamping time period. The motor control circuit 71 may be configured to estimate tissue thickness and/or tissue tension based at least in part on the motor torque (and/or clamping force and/or alternative motor parameter) during the clamping time period. A mathematical feature (e.g., time constant, initial torque or force, elapsed decay time, and/or other mathematical feature) of exponential decay of the motor torque (and/or clamping force and/or alternative motor parameter) during the clamping time period can correlate to tissue characteristics such as tissue type, tissue thickness, which may be used to learn ascertain insights of the tissue during a surgical procedure in real time. The motor control circuit 71 may further be configured to determine that the tissue property is undesirable and provide a user indication representing the undesirable tissue property. Examples of undesirable tissue property include calcification and staple line overlap. The motor control circuit 71 may further be configured to provide an estimation of the tissue property in real time.

In some embodiments, the motor control circuit 71 may be configured to utilize data related to motor torque (and/or clamping force and/or alternative motor parameter) during a previous clamping attempt affect a subsequent clamping attempt. For instance, if multiple unsuccessful clamping attempts are made, motor torque (and/or clamping force and/or alternative motor parameter) monitored during the unsuccessful clamping attempts can be used to modify the clamping speed of a subsequent clamping attempt and/or inform the user about tissue properties. The motor control circuit 71 can be configured to electrically drive the motor 63 through a first clamping time period, monitor the motor parameter of the motor 63 during the first clamping time period, and determine a clamping speed for a second clamping time period of a second clamping attempt based at least in part on the motor torque (and/or clamping force and/or alternative motor parameter) monitored during the first clamping time period.

In some embodiments, the motor control circuit 71 is configured to determine the end time of the clamping time period based at least in part on motor torque (and/or clamping force and/or alternative motor parameter) and manufacturing calibration parameters of the surgical instrument. For instance, in examples in which tissue compression is intended to reduce friction during a firing stroke following the clamping time period, the extent of tissue compression needed may depend on variations in friction experienced by the instrument during the firing stroke. Therefore, an instrument with higher friction during the firing stroke may require a lower threshold value F_e at the end of the clamping time period to achieve desired tissue compression. The clamping time period may therefore be longer for an instrument having a manufacturing calibration parameter that indicates higher inherent friction during a firing stroke.

In some embodiments, the motor control circuit 71 is configured to cause the motor 63 to pause at the end of a firing stroke prior to retracting the clamping/firing assembly 61 to a home position. The motor control circuit 71 can be configured to calculate the pause duration at the end of the firing stroke based at least in part on the motor parameter monitored during the clamping time period. The motor control circuit 71 can be configured to characterize tissue based on the motor parameter monitored during the clamping time period. The pause at end of firing prior to retraction can be precalculated, by the motor control circuit 71, based on the characterizations of the tissue during clamping.

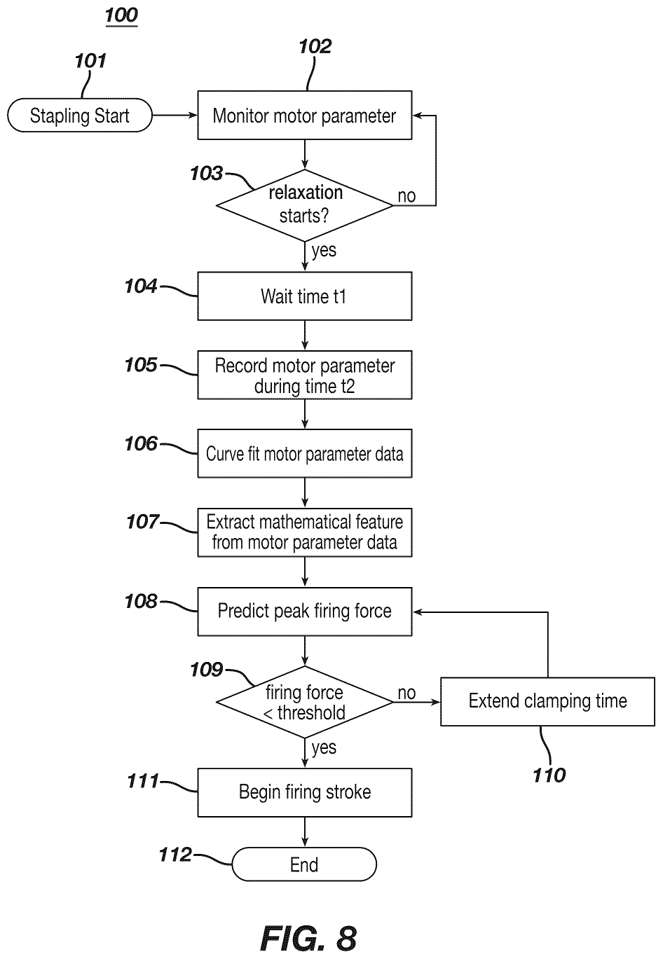

FIG. 8 is a flow diagram of a method 100 for a stapling procedure that includes clamping tissue for a clamping time period and deploying staples during a firing stroke following the clamping time period. The method 100 is suitable for a surgical instrument having a clamping/firing assembly including a knife at the distal end of the clamping/firing assembly. An example end effector 40 including a clamping firing assembly having a knife 43 is illustrated in FIG. 13. Example surgical systems, instruments, and devices presented herein having a clamping/firing assembly including a knife at the distal end of the clamping/firing assembly can further include one or more processors and memory in communication with the processor(s) such that the memory includes instructions executable by the processor(s) to cause the surgical instrument to perform steps of the method 100. For instance, the robotic surgical stapler system 15 illustrated in FIG. 1, alternatives thereto and variations thereof as understood by a person skilled in the pertinent art, can include a one or more processors and memory in communication with the processor(s) such that the memory includes instructions executable by the processor(s) to cause the surgical instrument to perform steps of the method 100. As another example, the handheld powered surgical stapler 10 illustrated in FIG. 2, alternatives thereto and variations thereof as understood by a person skilled in the pertinent art, can include a one or more processors and memory in communication with the processor(s) such that the memory includes instructions executable by the processor(s) to cause the surgical instrument to perform steps of the method 100. As another example, a surgical instrument having the end effector drive system 60 illustrated in FIG. 6, alternatives thereto and variations thereof as understood by a person skilled in the pertinent art, can include a one or more processors and memory in communication with the processor(s) such that the memory includes instructions executable by the processor(s) to cause the surgical instrument to perform steps of the method 100. For instance, the motor control circuit 71 can include one or more processors and memory in communication with the processor(s) such that the memory includes instructions executable by the processor(s) to cause the surgical instrument to drive the motor assembly 78 to perform steps of the method 100. The concepts of the method 100 can be applied to surgical instruments configured for a variety of surgical procedures which benefit from reliable compression of tissue such as energy tools and surgical grasping tools as understood by a person skilled in the pertinent art; as such, the method 100 is not limited solely to surgical stapling or cutting tools.

At block 101, stapling starts by a motor being driven to cause the clamping/firing assembly to engage and/or actuate the end effector. For instance, a surgical instrument equipped with the end effector drive system 60 illustrated in FIG. 6 can include instructions stored in memory of the motor control circuit 71 to cause the motor control circuit 71 to drive the motor assembly 78 and translate the clamping assembly 61 (which is a clamping/firing assembly) in the distal direction DD.

At block 102, knife force is monitored. For instance, a force provided by the motor 63 (FIG. 6) to drive the clamping/firing assembly to translate the knife 43 (FIG. 13) distally can be monitored. At block 102, the clamping/firing assembly is engaging with the end effector 40, and the jaws 41, 42 have not yet engaged tissue TT (FIG. 3).

At block 103, a determination is made as to whether or not relaxation of tissue has started. While the jaws 41, 42, have not yet engaged the tissue TT (FIG. 3), the method 100 continues to monitor knife force at block 102. Once it has been determined that the jaws 41, 42 have engaged tissue TT, the method proceeds to block 104. The determination can be made based at least in part on a distance of travel of the clamping/firing assembly, change in motor torque/knife load indicative of tissue engagement and/or onset tissue relaxation, increase in motor torque/knife load over a predetermine threshold, and/or based at least in part on sensor measurements that indicate tissue contact. For instance, it can be determined that clamping has started when the anvil 41 has rotated through a predetermined angle toward the staple jaw 42 (FIG. 3). As another example, it can be determined that clamping has started when the knife 43 has traveled a predetermined distance in the distal direction DD (FIG. 13), as another example, it can be determined that clamping has started when a transient signal of a motor parameter has been detected such as a transient in motor current, motor torque, motor voltage, etc. such that the transient indicates that the clamping/firing assembly has encountered a sudden change in mechanical resistance (e.g., friction).

At block 104, the method waits a time t1 in which the knife force is not utilized by the method. The clamping action continues at block 104, and the method waits until the elapse of time t1 before continuing to monitor the knife force. During time t1, the clamping force and/or motor torque may follow the curve in the initial time period t1 illustrated in the chart in FIG. 7. The initial time period t1 may include transients in the knife force data. The method 100 omits, or does not monitor, knife force data during the wait time t1, so that the knife force data can more precisely modeled in later portions of the clamping time period. In one example, during the wait time t1, the motor control circuit 71 drives the motor 63 to engage the clamping assembly 61 (FIG. 6) and begin closure of the jaws 41, 42 of the end effector 40 (FIG. 3). During the wait time t1, the clamping force and/or motor torque is likely to experience transients due to engagement of the motor 63, tissue TT, and components of the clamping assembly 61. The initial portion t1 can be set to have a predetermined duration. Alternatively, the end of the initial portion t1 can triggered based on an instrument parameter such as reaching a predetermine threshold value (e.g. torque/force value F1_max illustrated in FIG. 7).

At block 105, motor parameter (e.g. motor torque, clamping force, motor speed, or alternative parameter) is recorded during a predictive portion t2 of a clamping time. The predictive portion t2 begins after the initial transients in motor torque and clamping force have passed. The predictive portion t2 can be characterized as a portion of the clamping time period in which the knife force fits well to a curve with an exponential decay. For instance the clamping force and/or motor torque can have an exponential decay during the predictive portion t2 as illustrated in FIG. 7.

At block 106, the motor parameter data recorded at block 105 is mathematically modeled by fitting to a curve. For instance, the motor parameter data can be fit to curve with an exponential decay.

At block 107, a mathematical feature is extracted from the motor parameter data based on the mathematical model generated at block 106. For instance, the extracted mathematical feature can include a time constant, initial torque or force, elapsed decay time, and/or other parameter of an exponential decay curve that was fit, at block 106, to the motor parameter data, which was recorded, at block 105 during the predictive portion t2 of the clamping time period. The extracted mathematical feature can provide an indication of tissue compression.

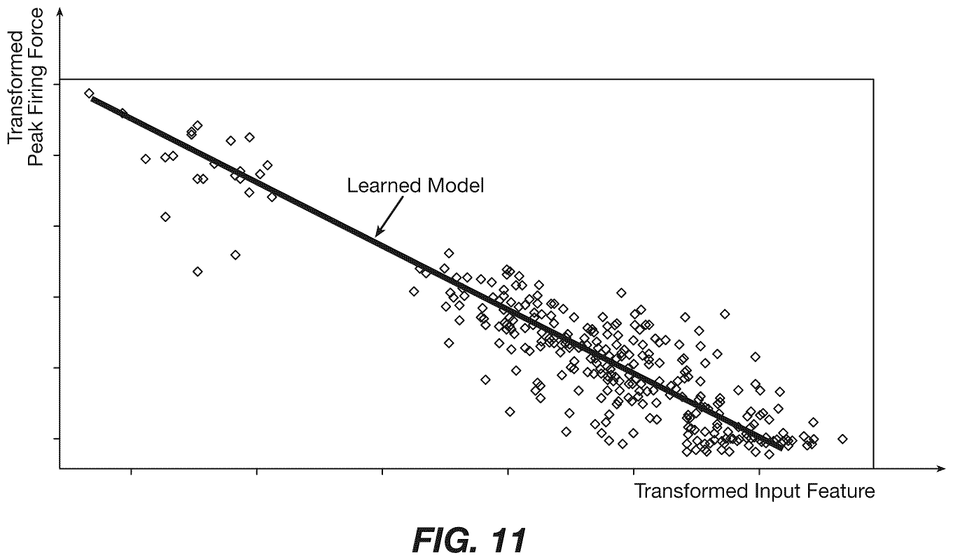

At block 108, a peak firing force can be predicted. The predicted peak firing force is an estimation of firing force that is expected during a firing stroke following the clamping time period. The peak firing force is predicted during the clamping time period, and therefore prior to initiating said firing stroke. Peak firing force is expected to be greater for tissue that has had less to time relax during compression and also tissue that is more resistant to compression. The predicted peak firing force is therefore a function of the tissue's resistance to compression and the total time that the tissue is compressed prior to the firing stroke. The feature extracted from the motor parameter data at block 107 provides an indication of the tissue's resistance to compression. For instance, the time constant of the exponential decay curve extracted at block 107 can provide an indication of the tissue's resistance to compression. Tissue stiffness may be determined based at least in part on the initial torque or force. Viscoelastic behavior may be determined by decay time constant and/or elapsed decay time. Peak firing force may also be a function of firing mechanism designs (assume limited variation for locked designs), tissue characteristics (e.g., type, stiffness, thickness) and relaxation time. Embodiments of the present disclosure may use a predication model which relies on empirical data fitted/learned from tests data. For instance, FIG. 11 illustrates an initial predication model learned from lab test data, which can be updated as more data/tests are obtained.

At block 109, the predicted peak firing force is compared to a firing force threshold. If the predicted peak firing force is greater than the firing force threshold, the method 100 proceeds to block 110. If the predicted peak firing force is less than the firing force threshold, the method 100 proceeds to block 111.

At block 110, the clamping time period is extended to allow the tissue additional time to relax under compression. Further relaxing the tissue under compression is expected to reduce the predicted peak firing force. Once the clamping time is extended, a new peak firing force is predicted at block 108. This new peak firing force is compared to the firing force threshold at block 109.

At block 111, the firing stroke may begin. The firing stroke may begin automatically. Alternatively, the surgical instrument may provide an indication to the user that they may engage a firing stroke, and the firing stroke may be engaged by the user.

At block 112, the method ends; however, the method may include additional steps related to utilizing the extracted feature (block 107) to set parameters of the firing stroke, set parameters of future clamping attempts, etc. as disclosed elsewhere herein.

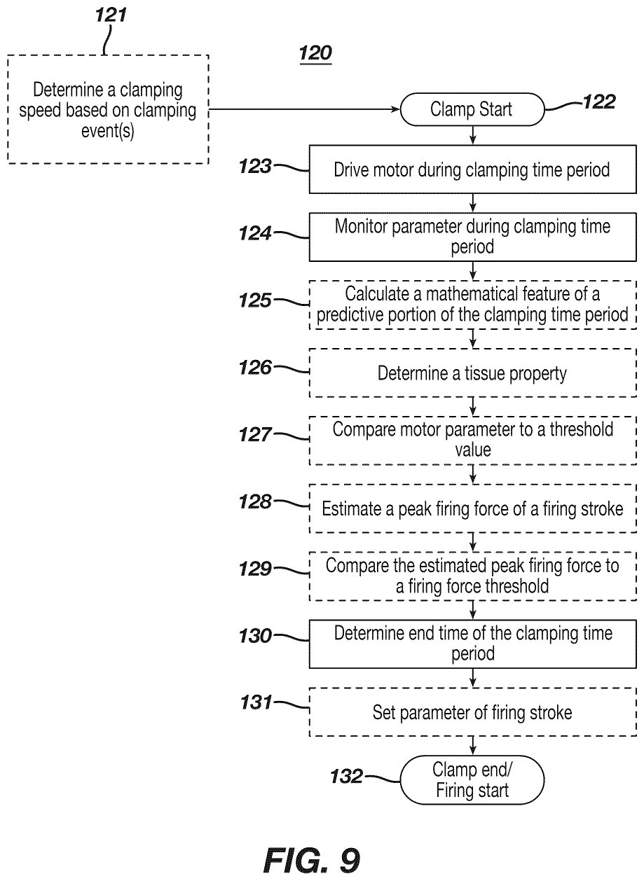

FIG. 9 is a flow diagram of a method 120 for controlling clamping time during a clamping time period. The method 120 is suitable for a variety of surgical instruments that clamp tissue. Example surgical systems, instruments, and devices presented herein can include one or more processors and memory in communication with the processor(s) such that the memory includes instructions executable by the processor(s) to cause the surgical instrument to perform steps of the method 120. For instance, the robotic surgical stapler system 15 illustrated in FIG. 1, alternatives thereto and variations thereof as understood by a person skilled in the pertinent art, can include a one or more processors and memory in communication with the processor(s) such that the memory includes instructions executable by the processor(s) to cause the surgical instrument to perform steps of the method 120. As another example, the handheld powered surgical stapler 10 illustrated in FIG. 2, alternatives thereto and variations thereof as understood by a person skilled in the pertinent art, can include a one or more processors and memory in communication with the processor(s) such that the memory includes instructions executable by the processor(s) to cause the surgical instrument to perform steps of the method 120. As another example, a surgical instrument having the end effector drive system 60 illustrated in FIG. 6, alternatives thereto and variations thereof as understood by a person skilled in the pertinent art, can include a one or more processors and memory in communication with the processor(s) such that the memory includes instructions executable by the processor(s) to cause the surgical instrument to perform steps of the method 120. For instance, the motor control circuit 71 can include one or more processors and memory in communication with the processor(s) such that the memory includes instructions executable by the processor(s) to cause the surgical instrument to drive the motor assembly 78 to perform steps of the method 120. As another example, the surgical instrument can be configured for a variety of surgical procedures which benefit from reliable compression of tissue such as energy tools and surgical grasping tools. Such surgical instruments can include one or more processors and memory in communication with the processor(s) such that the memory includes instructions executable by the processor(s) to cause the surgical instrument to perform steps of the method 120.

At optional block 121, a clamping speed can be determined based on previous clamping events. For instance, if one or more prior clamping events have occurred, the clamping speed for a subsequent clamping event can be adjusted so that the tissue can be more effectively clamped. For instance, clamping speed may be reduced to allow slower compression of thick or less compliant tissue. As another example, clamping speed may be increased to allow faster surgical time for treatment of thin or more compliant tissue. In some examples, the clamping speed can be adjusted in response to multiple unsuccessful clamping attempts.

At block 122, clamping can start.

At block 123, a motor can be driven during a clamping time period. For instance, motor 63 can drive clamping assembly 71 (FIG. 6) to cause jaws 41, 42 of the end effector 40 to close on tissue TT (FIG. 3).

At block 124, a motor parameter can be monitored during the clamping time period. The motor parameter can include a motor torque or alternative parameter as disclosed herein or otherwise understood by a person skilled in the pertinent art. The motor parameter need not be monitored during the entirety of the clamping time period. For instance, the clamping time period may include an initial portion (e.g. closure time period or initial portion of relaxation time period t1 as shown in FIG. 7) in which the motor parameter is not monitored.

At optional block 125, a time constant of a predictive portion of the clamping time period can be calculated. For instance, the motor parameter can decay exponentially during the predictive portion of the clamping time period and a time constant of the exponential decay can be calculated. The time constant may be calculated based on curve fitting or other mathematical model of the motor parameter during the predictive portion of the clamping time period. Additionally, or alternative to calculating the time constant, a different parameter can be utilized such as an initial torque of the motor parameter or a calculated elapsed decay time of the motor parameter during the predictive portion of the clamping time period.

At optional block 126, a tissue property can be determined. In some examples, the characteristics of force/torque decay during clamping can be used to estimate tissue properties for insights, such as tissue thickness, tissue tension, etc. For example, the time constant (and/or alternative mathematical feature) extracted from the curve-fitted exponential decay model correlates tissue characteristics such as tissue thickness or tissue type, which may be used to learn the insights of tissue during procedure in real-time. For instance, the motor control circuit 71 (FIG. 6) can be configured to estimate a tissue property based at least in part on the motor parameter monitored at block 124. The tissue property can include tissue thickness and/or tissue tension. The motor control circuit 71 can be configured to estimate the tissue property based at least in part on a time constant (and/or alternative mathematical feature such as initial torque, elapsed decay time, etc.) of an exponential decay model of the motor parameter during the clamping time period. Further, it may be determine that the tissue property is undesirable. A user indication may be provided such that the user indication represents the undesirable tissue property. The tissue property may be provided in real time.

At optional block 127, the motor parameter can be compared to a threshold value. For instance, the motor parameter may include a motor torque that is compared to a motor torque threshold. An elapsed time required to reach the motor torque threshold can be estimated.

At optional block 128, a peak firing force of a firing stroke can be estimated. The surgical instrument can be configured to engage the firing stroke following the clamping time period. The peak firing force expected to occur during the firing stroke can be estimated based at least in part on the motor parameter monitored during the clamping time period.

At optional block 129, the estimated peak firing force (block 128) can be compared to a firing force threshold.

At block 130, an end time of the clamping time period can be determined. For instance, the clamping time period can be extended beyond the predictive portion (block 125) of the clamping time period to the end time. The end time can be determined based at least in part on the motor parameter monitored at block 124. Additionally, or alternatively, the end time can be determined based at least in part on the previous clamping events at block 121. Additionally, or alternatively, the end time can be determined based on a time constant (and/or alternative mathematical feature) of a portion of the clamping time period as calculated in block 125. Additionally, or alternatively, the end time can be determined based at least in part on a tissue property determined at block 126. Additionally, or alternatively, the end time can be determined based at least in part on a comparison of the motor parameter (e.g. motor torque) to a threshold value (e.g. motor torque threshold). Additionally, or alternatively, the end time can be determined based at least in part on the estimated elapsed time for the motor parameter to reach a threshold value estimated at block 127. Additionally, or alternatively, the end time can be determined based at least in part on the comparison of the estimated peak firing force to the firing force threshold at block 129. Additionally, or alternatively, the end time can be determined based at least in part on the motor parameter and manufacturing calibration parameters of the surgical instrument.

At optional block 131, a parameter of a firing stroke can be set. For instance, a target speed, torque threshold, or other such firing stroke parameter as understood by a person skilled in the pertinent art can be set based at least in part on the motor parameter monitored during the clamping time period. For instance, a mathematical feature (e.g. an exponential decay value) of force or motor torque during a predictive portion (e.g. during a predictive portion of tissue relaxation time period) of a clamping time period can be used to set a target speed or motor torque threshold during the firing stroke that occurs once the clamping time period ends. As another example, the surgical instrument can include multiple firing stroke algorithms stored in memory, and the mathematical feature can be used to select one of the stored firing stroke algorithms. In this example, the parameter of the firing stroke is set by virtue of the selection of the firing stroke algorithm.

At block 132, the clamping time period ends. In some examples, a subsequent operation of the surgical instrument can commence automatically. For instance, a surgical instrument can automatically begin a firing stroke upon completion of the clamping time period. Alternatively, the surgical instrument may provide an indication to the user corresponding to the end of the clamping time period to inform the user that the clamping time period has elapsed. Additionally, or alternatively, the surgical instrument may include lockout features to prevent the subsequent operation of the surgical instrument before the clamping time period has ended. In some embodiments, the firing stroke operates using the parameter set at optional block 131.

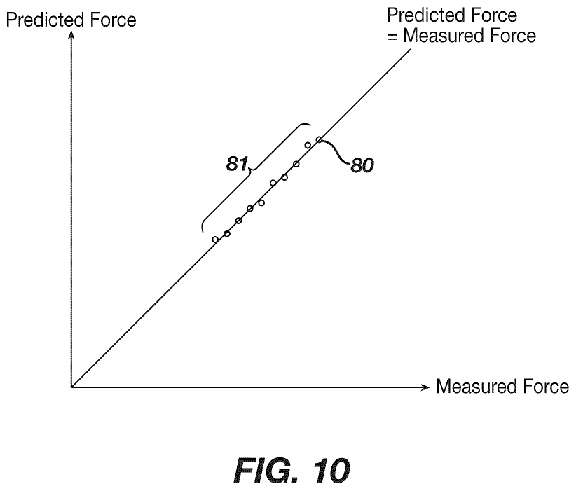

FIG. 10 is a chart illustrating predicted force correlated with measured force using a learned/curve fitted exponential decay model. A surgical stapler having a clamping/firing assembly configured similar to as illustrated in FIGS. 13 and 14A through 14D includes a motor that drives the clamping/firing assembly according to method 100 illustrated in FIG. 8 and/or the method 120 illustrated in FIG. 9. The predicted peak firing force is predicted according to steps described at block 108 of method 100 and/or according to step 125 of method 120. The firing stroke is initiated at block 111 of method 100 and/or step 132 of method 120, and the peak force during the firing stroke is measured. The data points 80 for predicted decay forces during clamping are plotted on the y-axis while the measured decay forces during clamping is plotted on the x-axis. The chart includes a line indicating where predicted clamping force is equal to the measured clamping force. The totality of the data 81 fits well to the line, indicating the clamping force response during tissue relaxation strictly follows exponential decay. The predicated force (y axis) is calculated from an exponential decay fitted model.

FIG. 11 is a chart illustrating transformed peak firing force as a function of a transformed input feature using a learned/curve fitted exponential decay model. This chart shows a linear curve fitted model learned from hundreds of tissue transection test data. The input (x-axis) is a mathematical feature (i.e., F_max in FIG. 7 during clamping period) mathematically transformed (for better curve fitting), and the output (y-axis) is peak firing force measured during firing period, which is also mathematically transformed (e.g., square root transformation) for better curve fitting. This chart indicates, through mathematical transformation, there is a relative clear linear correlation between a mathematical feature input (F_max) during clamping and the output peak firing force, which can be learned for peak firing force predication. The model may be improved/updated with more test data.

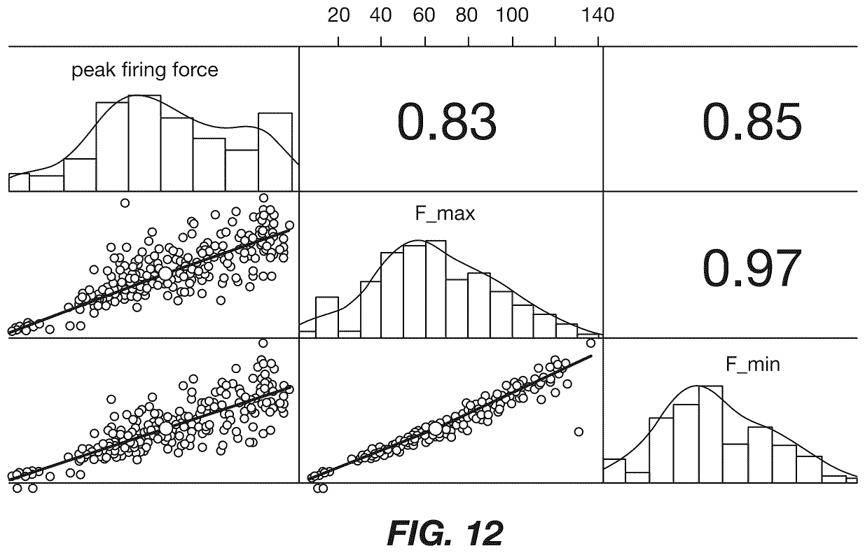

FIG. 12 illustrates a correlation matrix and raw data plotting of mathematical features (F_min, F_max referred in FIG. 3) extracted from decay of motor force/torque during a clamping time and the output parameter (peak firing force). In this chart (matrix), the diagonal cells show histogram distribution of the three parameters (i.e., peak firing force, F_max, F_min); the numbers in the upper right cells indicate the correlation coefficient of any pair those three parameters (e.g., 0.85 is the coefficient for F_min and peak firing force); and the left lower cells are the scatter plots (with linear fitted line) of any pair of those three parameters. This correlation matrix chart indicates that the features (F_max and F_min) extracted from clamping period are well correlated with the peak force during firing period. The correlation demonstrates that mathematical features of a motor parameter measured during a clamping time period can be used to predict peak firing force during a subsequent firing stroke.

FIG. 13 is an illustration of a sectional view of the end effector 40. The end effector 40 is an open position with the knife 43 at a home position XO prior to initiating clamping. A firing bar 31 extends in the proximal direction PD into the shaft 30. The firing bar 31 is translatable in the distal direction DD and the proximal direction PD. The knife 43 is positioned on a distal end of the firing bar 31. An I-beam 45 is coupled to the knife 43 and the distal portion of the firing bar 31. The firing bar pushes the I-beam 45 which has extensions that travel through an I-beam channel 55 of the anvil 41 and through an I-beam channel (not illustrated) of the staple jaw 42 to cause the anvil 41 to rotate toward the staple jaw 42 during the clamping time period and to maintain closure of the jaws 41, 42 during a firing stroke. During the clamping time period, the I-beam engages a shoulder 56 at a proximal end of the I-beam channel 55 through the anvil 41 to cause the anvil 41 to rotate toward the staple jaw 42.

The illustrated clamping assembly includes the firing bar 31 and I-beam 45. The firing bar 31 and I-beam 45, together with the knife 43, and a wedge sled 52 constitute a firing assembly that is can be driven by a motor assembly (e.g. motor assembly 78 in FIG. 6) during the firing stroke to deploy staples 51. The illustrated end effector 40 therefore has a clamping/firing assembly.

FIGS. 14A through 14D are a sequence of illustrations of a clamping assembly of an end effector during a clamping time period. The clamping assembly illustrated in FIGS. 14A through 14D operates similarly to the clamping assembly illustrated in FIG. 13. The firing bar 31, knife 43, and wedge sled 52 are not illustrated for the sake of simplicity. Similar to as illustrated in FIG. 13, the I-beam 45 includes extensions that engage an I-beam channel 57 in the staple jaw 42 and an I-beam channel 55 in the anvil 41. The I-beam channel 55 in the anvil 41 has a shoulder 56.

FIG. 14A shows the anvil in an open position similar to as illustrated in FIG. 13.

FIG. 14B shows the I-beam engaging the shoulder 56 of the I-beam channel 55 of the anvil 41. The I-beam 56 may experience transient forces upon engagement of the shoulder 56. The motor control circuit 71 need not record data related to knife force, tissue compression force, motor torque, or alternative motor parameter until after the transients have subsided.

FIG. 14C shows that the anvil 41 has closed to a small enough angle O so that tissue is engaged. FIG. 14C represents the beginning of the predictive period t2 of the clamping time period (FIG. 7).

FIG. 14D shows that as the I-beam 45 continues to travel distally during the clamping time period, the angle O between the anvil 41 and the staple jaw 42 continues to reduce. Accordingly, the tissue between the anvil 41 and staple jaw 42 compresses and reduces in thickness as illustrated in FIGS. 4A, 4B, 5A, and 5B. As the I-beam 45 traverses distally from the position illustrated in FIG. 14C to the position illustrated in FIG. 14D, the force on the tissue decays exponentially, and motor torque decays exponentially.

At the conclusion of the clamping time period, the I-beam can continue traveling distally through a firing stroke, preferably at a significantly faster speed.

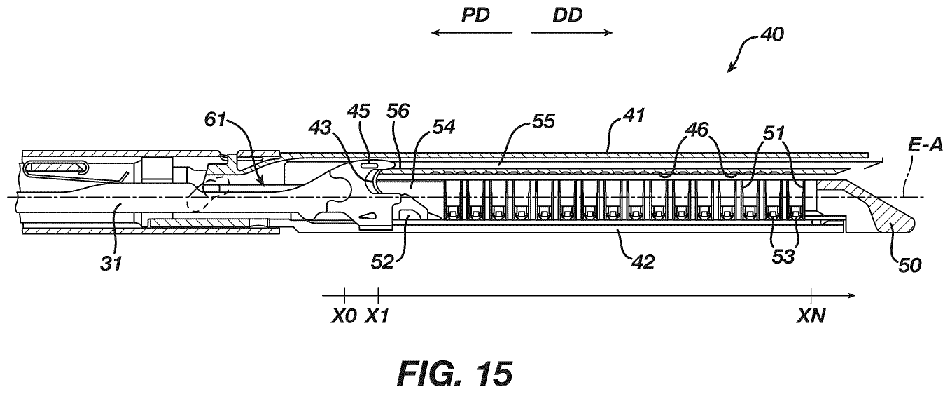

FIG. 15 shows the end effector 40 of FIG. 13 in a closed position. The knife 43 has moved from the home position XO to an initial position XI of the firing stroke. During the firing stroke, the I-beam 45 translates distally and the cutting edge of the knife 43 contacts and may cut tissue positioned between the anvil 41 and the staple cartridge 50. Also, the I-beam 45 contacts the wedge sled 52 and pushes it distally, causing the wedge sled 52 to contact staple drivers 53. The staple drivers 53 may be driven up into staples 51, causing the staples 51 to advance through tissue and into pockets 46 defined in the anvil 41, which shape the staples 51. The knife 43 is positioned at a distal position XN upon completion of the firing stroke. Portions or all of the clamping/firing assembly including the push rod 31 and I-beam 45 can be retracted proximally following the firing stroke.

Referring collectively to FIGS. 6 and 13-15, the control circuit 64 may be configured to set one or more target speeds during the firing stroke, and the motor control 65 may be configured to drive the motor 63 to the target speed(s) during the firing stroke. The target speed(s) set for the firing stroke (and other parameters such as acceleration, firing stroke length segmentation, etc.) may be determined as a function of the instrument data collected during at least a portion of the clamping time period. For example, the knife acceleration and initial target velocity can be reduced through a function informed by the decayed clamp force value prior to firing. In some examples the motor control circuit 71 is configured to set a parameter of the firing stroke (e.g. target acceleration and/or target velocity) based at least in part on the motor parameter of the motor during the clamping time period. The firing stroke comprises a plurality of segments over a firing stroke length, and the motor control circuit can be configured to set a first parameter of the firing stroke for a first segment of the plurality of segments and set a second parameter of the firing stroke for a second segment of the firing stroke of the plurality of segments.

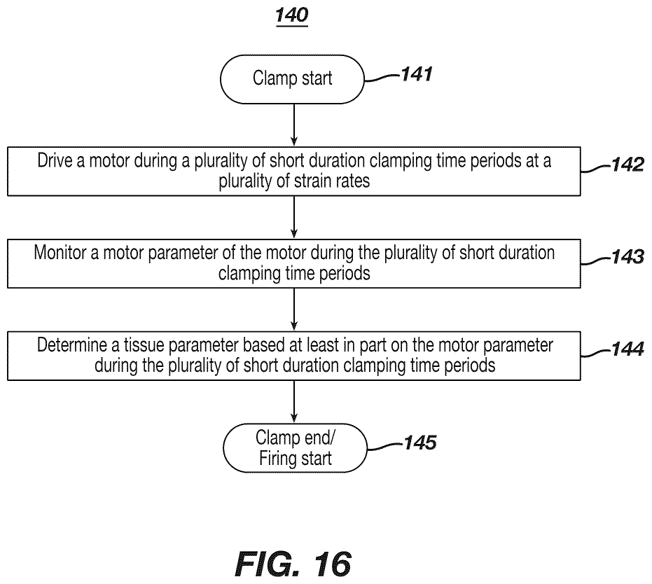

FIG. 16 is a flow diagram of another method 140 for controlling clamping time during a clamping time period. As an alternative to constant velocity clamping shown in FIG. 7 and disclosed elsewhere herein, closure of the jaws 41, 42 can include micro clamp and relaxation profiles in which a sequence of short clamping time periods, with pauses in between, are provided over a total clamping time period to close the jaws 41, 42 over tissue in preparation for a subsequent operation such as a firing stroke. The short clamping time periods can be applied at various strain rates to build up a more complex tissue characterization profile for short duration loading. This short duration loading profile can model short duration loading experienced during staple forming in a firing stroke.

The method 140 is suitable for a variety of surgical instruments that clamp tissue. Example surgical systems, instruments, and devices presented herein can include one or more processors and memory in communication with the processor(s) such that the memory includes instructions executable by the processor(s) to cause the surgical instrument to perform steps of the method 140. For instance, the robotic surgical stapler system 15 illustrated in FIG. 1, alternatives thereto and variations thereof as understood by a person skilled in the pertinent art, can include a one or more processors and memory in communication with the processor(s) such that the memory includes instructions executable by the processor(s) to cause the surgical instrument to perform steps of the method 140. As another example, the handheld powered surgical stapler 10 illustrated in FIG. 2, alternatives thereto and variations thereof as understood by a person skilled in the pertinent art, can include a one or more processors and memory in communication with the processor(s) such that the memory includes instructions executable by the processor(s) to cause the surgical instrument to perform steps of the method 140. As another example, a surgical instrument having the end effector drive system 60 illustrated in FIG. 6, alternatives thereto and variations thereof as understood by a person skilled in the pertinent art, can include a one or more processors and memory in communication with the processor(s) such that the memory includes instructions executable by the processor(s) to cause the surgical instrument to perform steps of the method 140. For instance, the motor control circuit 71 can include one or more processors and memory in communication with the processor(s) such that the memory includes instructions executable by the processor(s) to cause the surgical instrument to drive the motor assembly 78 to perform steps of the method 140. As another example, the surgical instrument can be configured for a variety of surgical procedures which benefit from reliable compression of tissue such as energy tools and surgical grasping tools. Such surgical instruments can include one or more processors and memory in communication with the processor(s) such that the memory includes instructions executable by the processor(s) to cause the surgical instrument to perform steps of the method 140.

At block 141, clamping can start.

At block 142, a motor can be driven during a plurality of short duration clamping time periods at a plurality of strain rates. The plurality of short duration clamping time periods can be provided in succession during a total clamping time period such that each of the short duration clamping time periods provides a fraction of compression of tissue during the total clamping time period, and the succession of the plurality of short duration clamping time periods completes the process of clamping tissue prior to another operation such as a firing stroke. The strain rate can be different for at least two durations of the plurality of short duration clamping time periods.

At block 143, a motor parameter of the motor can be monitored during the plurality of short duration clamping time periods. The motor parameter can include a motor torque. Portions of each of the plurality of short duration clamping time periods that include transients need not be monitored. The motor parameter can be monitored similar to as disclosed in relation to block 102 of method 100 in FIG. 8, as disclosed in relation to block 124 of method 120 in FIG. 9, as disclosed elsewhere herein, and/or as otherwise understood by a person skilled in the pertinent art.

At block 144, a tissue parameter can be determined based at least in part on the motor parameter monitored at block 143. For instance, the motor parameter may include a predictive portion for at least some of the plurality of short duration clamping time periods such that the motor parameter can be mathematically modeled during the predictive time period. A time constant or other feature of the mathematical model can be used to determine the tissue parameter. For instance, at least some of the plurality of short duration clamping time periods can have a predictive portion that can be curve fit to a respective exponential decay, each with a respective time constant, initial torque or force, elapsed decay time, and/or other suitable mathematical feature. Tissue characteristics such as thickness, type, pliability, etc. can be calculated based at least in part on the strains and mathematical features (e.g. time constants) of the plurality of short duration clamping time periods.

The method 140 can further be modified to adjust the number of short duration clamping time periods based at least in part on the motor parameter of one or more subsequent short duration clamping time periods. Analysis of the motor parameter data collected during short duration clamping time periods may indicate that the tissue has been sufficiently compressed and that no additional clamping time periods are needed and the total clamping time period can end. For instance, the motor parameter can be compared to a threshold value, and the total clamping time period can end or continue based at least in part on the comparison. Additionally, or alternatively, analysis of the motor parameter data collected during short duration clamping time periods may indicate that the tissue requires additional compression, resulting in additional short duration clamping time periods before the total clamping time period can end. In some examples, characteristics of one or more subsequent short duration clamping time period(s) (e.g., target speed, strain, clamping duration, etc.) may be set based at least in part on the motor parameter data collected during at least one previous short duration clamping time period.

Analysis of the motor parameter data collected during short duration clamping time periods may involve estimating a peck firing force of a firing stroke. In some examples, the method 140 may include comparing the peek firing force to a firing force threshold and determining aspects of subsequent short duration clamping time periods based on the comparison. As such, the end time of the total clamping time period can be based at least in part on the comparison of the peck firing force to the firing force threshold. In some examples, characteristics of one or more subsequent short duration clamping time period(s) (e.g., target speed, strain, clamping duration, etc.) may be set based at least in part on the comparison of the peck firing force to the firing force threshold.

At block 145, the total clamping time period ends. In some examples, a subsequent operation of the surgical instrument can commence automatically. For instance, a surgical instrument can automatically begin a firing stroke upon completion of the clamping time period. Alternatively, the surgical instrument may provide an indication to the user corresponding to the end of the clamping time period to inform the user that the clamping time period has elapsed. Additionally, or alternatively, the surgical instrument may include lockout features to prevent the subsequent operation of the surgical instrument before the clamping time period has ended.

In some examples, aspects of the firing stroke (e.g. acceleration, speed, segmentation) can be determined based at least in part on at least some of the motor parameters monitored during the plurality of short duration clamping time periods.

The following clauses list non-limiting embodiments of the disclosure:

Clause 1. A surgical instrument (10, 11) comprising: an end effector (40) comprising a pair of jaws (41, 42); a motor assembly (63, 66) comprising a motor (66) mechanically coupled to the end effector (40), the motor assembly (13, 66, 67) being configured to actuate the end effector to grasp and compress tissue (TT) between the pair of jaws (41, 42); and a motor control circuit (71) configured to: electrically drive the motor (63) during a clamping time period, monitor a motor parameter of the motor (63) during the clamping time period, and determine an end time (t_end) of the clamping time period based at least in part on the motor parameter.

Clause 2. The surgical instrument of clause 1, wherein the motor parameter comprises a motor torque.

Clause 3. The surgical instrument of clause 2, wherein the motor torque is configured to exponentially decay through a predictive portion of the clamping time period.

Clause 4. The surgical instrument of clause 3, wherein motor control circuit (71) is configured to: calculate a mathematical feature of the motor torque through the predictive portion of the clamping time period, and determine the end time (t_end) based at least in part on the mathematical feature.

Clause 5. The surgical instrument of clause 3 or 4, wherein the motor control circuit (71) is configured to: drive the motor (63) through an initial predetermined time period (t1) of the clamping time period, drive the motor (63) through the predictive portion (t2) of the clamping time period, calculate a mathematical feature of the motor torque through the predictive portion (t2) of the clamping time period, estimate an elapsed time required to reach a motor torque threshold, and determine the end time (t_end) based at least in part on the estimated elapsed time.

Clause 6. The surgical instrument of clause 4 or 5, wherein the mathematical feature comprises one or more of a time constant, an initial force, an initial torque, or an elapsed decay time.

Clause 7. The surgical instrument of any one of clauses 4-6, wherein the mathematical feature comprises a time constant.

Clause 8. The surgical instrument of any one of clauses 2-7, wherein the motor control circuit is configured to: compare the motor torque to a motor torque threshold, and determine the end time based (t_end) at least in part on the comparison of the motor torque to the motor torque threshold.

Clause 9. The surgical instrument of any one of clauses 1-8, wherein the pair of jaws comprises an anvil (41) and a staple jaw (42), wherein the end effector (40) is configured to deploy staples (51) into the tissue (TT) during a firing stroke, and wherein the motor control circuit (71) is configured to estimate a peak firing force during the firing stroke based at least in part on the motor parameter monitored during the clamping time period.

Clause 10. The surgical instrument of clause 9, wherein the motor control circuit (71) is configured to: compare the estimated peak firing force to a firing force threshold, and determine the end time (t_end) based at least in part on the comparison of the estimated firing force to the firing force threshold.

Clause 11. The surgical instrument of any one of clauses 1-10, wherein the pair of jaws comprises an anvil (41) and a staple jaw (42), wherein the end effector (40) is configured to deploy staples (51) into the tissue (TT) during a firing stroke, and wherein the motor control circuit (71) is configured to initiate the firing stroke in response to the clamping time period reaching the end time (t_end).

Clause 12. The surgical instrument of any one of clauses 1-11, comprising a robotic surgical stapler (11) comprising the end effector (40), the motor assembly (63, 66, 13), and the motor control circuit (71).

Clause 13. The surgical instrument of any one of clauses 1-11, comprising a handheld surgical stapler (10) comprising the end effector (40), the motor assembly (63, 66), and the motor control circuit (71).

Clause 14. The surgical instrument of any one of clauses 1-11, wherein the pair of jaws comprises an anvil (41) and a staple jaw (42), wherein the end effector (40) is configured to deploy staples (51) into the tissue (TT) during a firing stroke, and wherein the motor control circuit (71) is configured to set a parameter of the firing stroke based at least in part on the motor parameter of the motor during the clamping time period.

Clause 15. The surgical instrument of clause 14, wherein the motor control circuit (71) is configured to set the parameter of the firing stroke such that the parameter of the firing stroke includes a target acceleration and/or a target velocity.

Clause 16. The surgical instrument of clause 14 or 15, wherein the firing stroke comprises a plurality of segments over a firing stroke length, and wherein the motor control circuit is configured to set said parameter of the firing stroke for a first segment of the plurality of segments and set a second parameter of the firing stroke for a second segment of the firing stroke of the plurality of segments.

Clause 17. The surgical instrument of any one of clauses 1-16, wherein the motor control circuit (71) is configured to estimate a tissue property based at least in part on the motor parameter of the motor during the clamping time period.

Clause 18. The surgical instrument of clause 17, wherein the tissue property comprises tissue thickness and/or tissue tension.

Clause 19. The surgical instrument of clause 17 or 18, wherein the motor control circuit (71) is configured to estimate the tissue property based at least in part on a time constant of an exponential decay model of the motor parameter during the clamping time period.

Clause 20. The surgical instrument of clause 19, wherein the mathematical feature comprises one or more of a time constant, an initial force, an initial torque, or an elapsed decay time.

Clause 21. The surgical instrument of claim 19 or 20, wherein the mathematical feature comprises a time constant.

Clause 22. The surgical instrument of any one of clauses 17-21, wherein the motor control circuit is configured to: determine that the tissue property is undesirable, and provide a user indication representing the undesirable tissue property.

Clause 23. The surgical instrument of any one of clauses 17-22, wherein the motor control circuit (71) is configured to provide the estimation of the tissue property in real time.

Clause 24. The surgical instrument of any one of clauses 1-23, wherein the end effector (40) comprises electrodes configured to deliver thermal treatment to the tissue (TT).

Clause 25. The surgical instrument of any one of clauses 1-24, wherein said clamping time period is a second clamping time period, and wherein the motor control circuit is configured to: electrically drive the motor (63) during a first clamping time period, monitor the motor parameter of the motor (63) during the first clamping time period, and determine a clamping speed associated with the second clamping time period based at least in part on the motor parameter during the first clamping time period.

Clause 26. The surgical instrument of any one of clauses 1-25, wherein the motor control circuit is configured to determine the end time of the clamping time period based at least in part on the motor parameter and manufacturing calibration parameters of the surgical instrument.

Clause 27. The surgical instrument of any one of clauses 1-26, wherein the pair of jaws comprises an anvil (41) and a staple jaw (42), wherein the end effector (40) is configured to deploy staples (51) into the tissue (TT) during a firing stroke, and wherein the motor control circuit (71) is configured to estimate a peak firing force during the firing stroke based at least in part on the motor parameter monitored during the clamping time period and manufacturing calibration parameters of the surgical instrument.

Clause 28. The surgical instrument of any one of clauses 1-27, wherein the pair of jaws comprises an anvil (41) and a staple jaw (42), wherein the end effector (40) is configured to deploy staples (51) into the tissue (TT) during a firing stroke, and wherein the motor control circuit (71) is configured to calculate a pause duration at the end of the firing stroke based at least in part on the motor parameter monitored during the clamping time period.

Clause 29. The surgical instrument of any one of clauses 1-28, further comprising: a shaft (30) extending along a longitudinal axis (S-A); an articulation joint (44) coupling the shaft (30) to the end effector (40) and configured to bend to angle the end effector (40) with respect to the longitudinal axis (S-A); and an articulation control circuit configured to modify the angle of the end effector (40) based at least in part on the motor parameter.

Clause 30. A surgical instrument (10, 11) comprising: an end effector (40) comprising a pair of jaws (41, 42); a motor assembly (63, 66) comprising a motor (66) mechanically coupled to the end effector (40), the motor assembly (13, 66, 67) being configured to actuate the end effector to grasp and compress tissue (TT) between the pair of jaws (41, 42); and a motor control circuit (71) configured to: electrically drive the motor (63) during a plurality of short duration clamping time periods at a plurality of strain rates, monitor a motor parameter of the motor (63) during the plurality of clamping time periods, and determine a tissue parameter based at least in part on the motor parameter during the plurality of clamping time periods.

Having shown and described exemplary embodiments of the subject matter contained herein, further adaptations of the methods and systems described herein may be accomplished by appropriate modifications without departing from the scope of the claims. For instance, software methods can be realized in various types of hardware; and software methods can include additional steps; the surgical stapler 10 can be modified to include alternative and/or additional compatible features of other surgical staplers known in the art or yet to be developed. In addition, where methods and steps described above indicate certain events occurring in certain order, it is intended that certain steps do not have to be performed in the order described but, in any order, as long as the steps allow the embodiments to function for their intended purposes. Therefore, to the extent there are variations of the invention, which are within the spirit of the disclosure or equivalent to the inventions found in the claims, it is the intent that this patent will cover those variations as well. Some such modifications should be apparent to those skilled in the art. For instance, the examples, embodiments, geometrics, materials, dimensions, ratios, steps, and the like discussed above are illustrative. Accordingly, the claims should not be limited to the specific details of structure and operation set forth in the written description and drawings.

1. A surgical instrument comprising:
an end effector comprising a pair of jaws;
a motor assembly comprising a motor mechanically coupled to the end effector, the motor assembly being configured to actuate the end effector to grasp and compress tissue between the pair of jaws; and
a motor control circuit configured to:
electrically drive the motor during a clamping time period,
monitor a motor parameter of the motor during the clamping time period, and
determine an end time of the clamping time period based at least in part on the motor parameter.

2. The surgical instrument of claim 1, wherein the motor parameter comprises a motor torque.

3. The surgical instrument of claim 2, wherein the motor torque is configured to exponentially decay through a predictive portion of the clamping time period.

4. The surgical instrument of claim 3, wherein the motor control circuit is configured to:
drive the motor through an initial predetermined time period of the clamping time period,
drive the motor through the predictive portion of the clamping time period,
calculate a mathematical feature of the motor torque through the predictive portion of the clamping time period,
estimate an elapsed time required to reach a motor torque threshold based at least in part on the mathematical feature, and
determine the end time based at least in part on the estimated elapsed time and/or the mathematical feature, wherein the mathematical feature comprises one or more of a time constant, an initial force, an initial torque, or an elapsed decay time.

5. The surgical instrument of claim 2, wherein the motor control circuit is configured to:
compare the motor torque to a motor torque threshold, and
determine the end time based at least in part on the comparison of the motor torque to the motor torque threshold.

6. The surgical instrument of claim 1,
wherein the pair of jaws comprises an anvil and a staple jaw,
wherein the end effector is configured to deploy staples into the tissue during a firing stroke, and
wherein the motor control circuit is configured to estimate a peak firing force during the firing stroke based at least in part on the motor parameter monitored during the clamping time period.

7. The surgical instrument of claim 1,
wherein the pair of jaws comprises an anvil and a staple jaw,
wherein the end effector is configured to deploy staples into the tissue during a firing stroke, and
where the motor control circuit is configured to initiate the firing stroke in response to the clamping time period reaching the end time.

8. The surgical instrument of claim 1,
wherein the pair of jaws comprises an anvil and a staple jaw,
wherein the end effector is configured to deploy staples into the tissue during a firing stroke, and
wherein the motor control circuitis configured to set a parameter of the firing stroke based at least in part on the motor parameter of the motor during the clamping time period.

9. The surgical instrument of claim 1, wherein the motor control circuit is configured to estimate a tissue property based at least in part on the motor parameter of the motor during the clamping time period.

10. The surgical instrument of claim 1, wherein the end effector comprises electrodes configured to deliver thermal treatment to the tissue.

11. The surgical instrument of claim 1, wherein said clamping time period is a second clamping time period, and
wherein the motor control circuit is configured to:
electrically drive the motor during a first clamping time period, monitor the motor parameter of the motor during the first clamping time period, and
determine a clamping speed associated with the second clamping time period based at least in part on the motor parameter during the first clamping time period.

12. The surgical instrument of claim 1, wherein the motor control circuit is configured to determine the end time of the clamping time period based at least in part on the motor parameter and manufacturing calibration parameters of the surgical instrument.

13. The surgical instrument of claim 1,
wherein the pair of jaws comprises an anvil and a staple jaw,
wherein the end effector is configured to deploy staples into the tissue during a firing stroke, and
wherein the motor control circuit is configured to estimate a peak firing force during the firing stroke based at least in part on the motor parameter monitored during the clamping time period and manufacturing calibration parameters of the surgical instrument.

14. The surgical instrument of claim 1,
wherein the end effector is configured to deploy staples into the tissue during a firing stroke, and
wherein the motor control circuit is configured to calculate a pause duration at the end of the firing stroke based at least in part on the motor parameter monitored during the clamping time period.

15. The surgical instrument of claim 1, further comprising:
a shaft extending along a longitudinal axis;
an articulation joint coupling the shaft to the end effector and configured to bend to angle the end effector with respect to the longitudinal axis; and
an articulation control circuit configured to modify the angle of the end effector based at least in part on the motor parameter.

16. The surgical instrument of claim 4, wherein the mathematical feature comprises a time constant.

17. The surgical instrument of claim 9, wherein the motor control circuit is configured to estimate the tissue property based at least in part on a mathematical feature of an exponential decay model of the motor parameter during the clamping time period.

18. The surgical instrument of claim 9, wherein the motor control circuit is configured to:
determine that the tissue property is undesirable, and
provide a user indication representing the undesirable tissue property.

19. The surgical instrument of claim 9, wherein the motor control circuit is configured to provide the estimation of the tissue property in real time.

20. A surgical instrument comprising:
an end effector comprising a pair of jaws;
a motor assembly comprising a motor mechanically coupled to the end effector, the motor assembly being configured to actuate the end effector to grasp and compress tissue between the pair of jaws; and
a motor control circuit configured to:
electrically drive the motor during a plurality of short duration clamping time periods at a plurality of strain rates,
monitor a motor parameter of the motor during the plurality of clamping time periods, and
determine a tissue parameter based at least in part on the motor parameter during the plurality of clamping time periods.
